(19) United States
(12) Patent Application Publication
(10) Pub. No.: US 2020/0279639 A1
(43) Pub. Date: Sep. 3, 2020

(54) NETWORK DISTRIBUTED SYSTEM FOR PEOPLE PAIRING AND EXECUTION OF TRAINING OR REHABILITATION SESSION

(71) Applicant: FIGHTTHESTROKE FOUNDATION, Milano (MI) (IT)

(72) Inventors: Francesca Fedeli, Milano (MI) (IT); Roberto D'Angelo, Milano (MI) (IT)

(21) Appl. No.: 16/645,917

(22) PCT Filed: Sep. 12, 2018
(86) PCT No.: PCT/IB2018/056965

§ 371 Date: Mar. 10, 2020

(30) Foreign Application Priority Data
Sep. 13, 2017 (IT) 102017000102346

(51) Int. Cl. G16H 40/20; G16H 20/30; G16H 20/70; G06T 7/00; G06F 16/2457; G06F 16/2455; G06F 16/28

(52) U.S. Cl. CPC G16H 40/20; G16H 20/30; G16H 20/70; G06F 16/284; G06F 16/24578; G06F 16/24564; G06T 7/0012

(57) ABSTRACT

A method for the coupling of subjects in a computerized system (10) for training or rehabilitation is described. According to the method it creates a user database (14) of subjects available be paired and assign to each subject all indicators that are useful in determining the pairings between subjects. Then, pairs of subjects are automatically formed in the user database (14) according to the rules of coupling memorized in a memory (26) and training or rehabilitation sessions are started among the pairs of subjects using user terminals (19). The images of each subject in the pair, during the sessions, is taken by a camera (20) and according to the images analyzed the system automatically check if the pair still meets the rules in the memory (26) or not and, in the case, it tries to form a new pair. Also a computational system for the execution of the method is described.

The present invention refers to an innovative method and network distributed system for the coupling of people, in particular for the purpose of using pairs of subjects in a “student/teacher-teacher/student” system, for example for training purpose and/or the rehabilitation of people. With “student/teacher-teacher/student system” here refers to a system where two subjects perform tasks as a student (i.e. learning the task to perform and try to reproduce it), but who share between them the information (even if only visual) needed to correct/improve each other, so that at least one of them also plays the role of “teacher” for the other user even for a part of the session.

In particular, the pairs of subjects can be formed by children who have suffered damage to the central nervous system, for example following a stroke, and who have a consequent motor deficit.

In these subjects, the typically proposed rehabilitation consists mostly in rehabilitation sessions, for example two or three a week, for prolonged periods (even years) and manually executed in dedicated physical rehabilitation centers and in the presence of a human operator, typically a specialized physiotherapist and with no standardized methods or data collection.

Whether it's two-hand rehabilitation, or constrictive or robotics or in virtual reality, whether it is for prolonged periods in years or intensive periods (typically a month), in each case, the patient and his family are forced to travel and support heavy costs (including missed productivity for the parents or missed school hours for the kids) to be able to take advantage of these therapies. Moreover, often the possibility of effective functional rehabilitation is undermined by the scarcity of the offer and of the rehabilitation centers. The experience of the individual therapist is also often essential for a satisfactory outcome.

The consequences are very high levels of stress for the whole ecosystem, non-standardized techniques, not-standardized data collection or, in some cases, exercises designed to improve the performance of a patient only in some metrics but not necessarily useful from the point of view of the patient's real quality of life.

To try to avoid travel costs of patients it was also proposed the use of tele-rehabilitation, namely the exercises are shown and monitored remotely from a therapist, which, however, remains always necessary for the entire duration of the session.

This is true not only in the case of need for rehabilitation but also in the case of sessions and training for various activities, such as training for sports activities, for the development of mental and physical abilities, to run physical exercises, diets, for old people support, etc.

Since the discovery of Mirror Neurons and brain plasticity, especially in the case of children, the emulation of exercises between the two subjects, appropriately paired to you, can greatly improve the effectiveness of any session. The latest findings in the field of neuroscience have in fact demonstrated the potential of Mirror Neurons in motor learning. Mirror Neurons are a class of neurons that is activated when an individual performs an action and when the individual observes the same action performed by another subject, provided that the action is already somehow present in the “skills domain” the person that is observing.

The training and rehabilitation techniques based on the activation of Mirror Neurons go under the name of Action Observation Treatment (or therapy according to the bibliography), relying on the capability of the brain to learn motor skills through the observation of some videos.

Also in this case, however, the clinical trial, for example in the case of rehabilitation techniques, have been focused on the figure of the therapist and very limited focus has been dedicated to the overall experience of the patient and his family (or caregivers) and always acting on a single patient at a time.

In any case, there is still the problem of having to employ a physiotherapist that in the case pairs two patients, makes them perform the rehabilitative exercises and verifies the results obtained during each session. The two patients, however, must also be present on the same place with the physiotherapist and will therefore have the same logistical problems early mentioned.

Similar problems can be found also in the case of training for activities different from rehabilitation and the system and the method according to the invention are suitable for coupling of subjects also for those other activities, as it will be clear in the following sections. The general purpose of the present invention is therefore to provide a method and a computational system that can automatically creates pairs of subjects, can monitor the progress of the sessions of subjects paired in order to verify the validity of the pair over time and possibly change the couplings when needed.

In view of this aim, it has been thought to realize, according to the invention, a method for the coupling of entities into a computerized system for training or rehabilitation, comprising the steps of:

a) create in a computerized system a user profile database of subjects to be paired, assigning to every subject values defined as useful indicators to determine the couplings between subjects;

b) establish and store in a memory of the computerized system rules for the coupling that define a permissible deviations between values of two subjects indicators in the user database in order to be paired;

c) automatically forming pairs of subjects in the database according with rules stored in the system memory and with the indicators associated to every subject and create a database in the computerized system of thus formed pairs;

d) starting, in the computerized system, of training or rehabilitation sessions between pairs of subjects in the database of torques via user terminals available to each person in the pair.

e) Analyze with a camera the images of each subject of the pair during the sessions and automatically calculate/update motor capability indexes lm and/or stress level ls of the subject according to the detected images;

f) calculating in the computer system, as a function of lm motor index and/or stress ls established for the subject, new values for this subject of the associated indexes useful in order to establish couplings between subjects;

g) automatically check if the new values of the indicators of the two subjects of the pair still satisfy the rules stored in the memory and in the event of a positive response maintain the torque, while in the event of a negative answer cancel the pair and try to form, based to the rules stored in the memory (26) and the new values of the indexes of the subjects of the canceled torque, a new torque between each subject of the canceled torque and other unmated subjects present in the user database.

Eventually, you can then repeat steps from d) to g).

It is also thought to realize, according to the invention, a computerized system for the coupling of subjects and the execution of training or rehabilitation sessions between these subjects according to the method, comprising a remote server connected to a plurality of user posts, user database, pair database and memory.

To make clearer the explanation of the innovative principles of the present invention and its advantages over the prior techniques, it will be described hereinafter, with the help of the accompanying drawings, an exemplary embodiment applying such principles.

In the drawings:

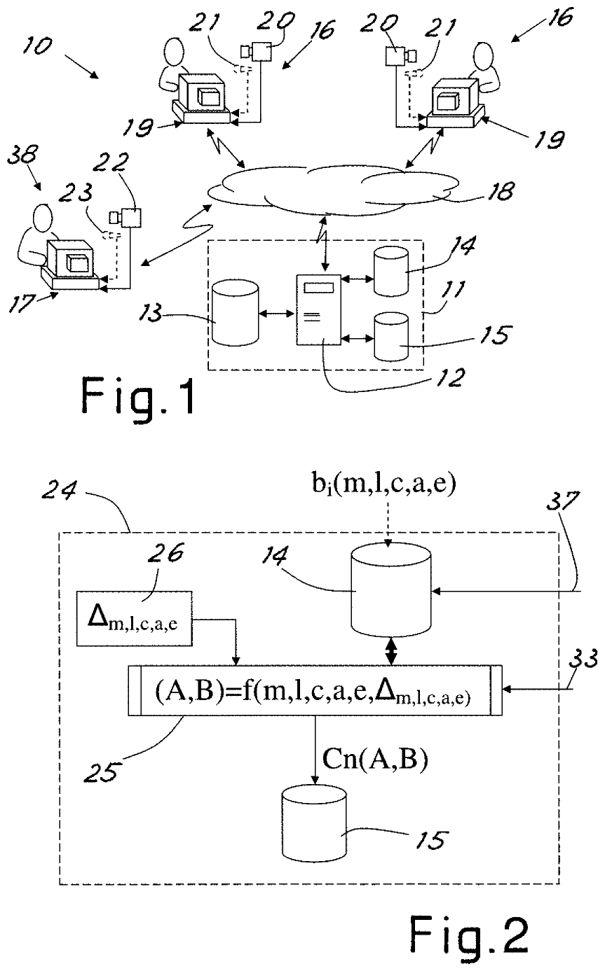

FIG. 1 shows a schematic view of the structure of a computerized distributed system for the coupling, verification and eventual recoupling of the subjects, in particular patients (for example, children with motor impairments and/or cognitive deficits) that execute remote rehabilitation sessions with the use of terminals that display the tasks to be performed.

FIG. 2 represents a block diagram in greater detail of a part of the system in FIG. 1;

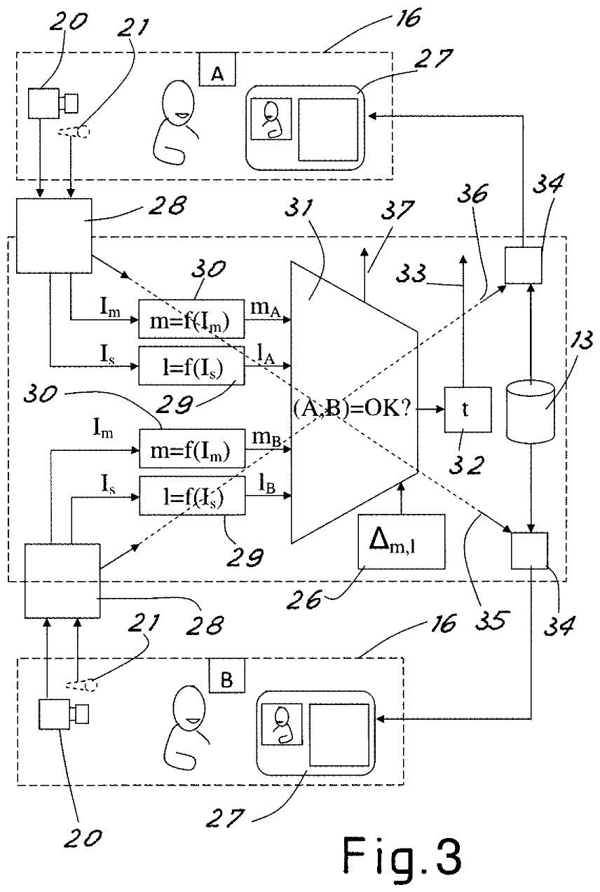

FIG. 3 shows a block diagram in greater detail of another part of the system in FIG. 1.

With reference to the figures, FIG. 1 shows a computerized system, generally indicated with 10, which includes a server 11. The server 11 is in remotely connected to a plurality of user posts 16 (of which two are shown in FIG. 1). It can also be provided with one or more supervisor workstations 38 in which a professional operator can supervise, correct and enter data into the system, as will be clear in the following sections.

The user posts 16, the optional supervisor workstation 38 and the server 11 can be connected in a network 18, for example the Internet network.

The server 11 is advantageously provided with a processing unit 12 and a database 13, 14 and 15 which respectively contain multimedia content M (for example, explanatory films of exercises that users must fulfill), user data U that use or must use the system, and data C of the pairs formed by users.

The user posts 16 include a terminal 19, a camera 20 and a microphone 21. The terminal 19 includes an electronic control unit with a display device and possibly an input device of which a keyboard, a joystick, a mouse, etc.

As will be clear from the following, cameras can be 2D (standard webcam) or 3D, and be part of a system for facial expressions and motion detection (for example as in the Microsoft Kinect or Intel Realsense).

The terminal 19 can be constituted for example by a personal computer with a custom application, by a dedicated system with monitor and keyboard or by a known system with a processing unit (such as a gaming console, a smartTV or a mobile device with a custom application) connected to a TV or any large screen. These and other solutions will be easily imaginable by the technician on the basis of the description made here.

Even the eventual supervisor station 38 may have similar structure as the user station. However, it has been found advantageous that the supervisor terminal 17 of the supervisor station 38 is realized with a personal computer suitably programmed. The supervisor station can also include a camera 22 and a microphone 23.

In FIG. 2 it is shown a block diagram of a part 24 of the system 10 inserted into the server 11 and that creates the database of the pairs C 15 starting from the database of the users U 14.

Initially, the user database 14 is created with data of subjects who need to use the system. In particular, such data may be advantageously inserted by one or more operators through one or more supervisor terminals 17. During this operation, the supervisors terminals can also be normal terminals for data entry, such as personal devices connected to the system, even temporarily, through a remote network connection (through the Internet or an intranet).

The data entered for each user must contain an identifier of the user (for example, a User Name, name and surname of the user, address, etc.) and all the indicators useful for establishing the connections between users.

For example, the indicators may have assigned values which establish a condition of the psycho-physical state of the subjects.

These indicators can be a motor index and/or a stress or emotional index, as will be clear from the following.

For example, in the case that the users are patients undergoing to a rehabilitation, the indicators may have assigned values that determine peculiarities of the psycho-physical state of the various patients.

Still more in particular, in the case of patients with motors and/or cognitive problems, such as stroke patients (especially children), it has been found to be advantageous that for each patient bᵢ there are associated at least three indicators, namely m=motor index, l=leadership index, c=cognitive index, and preferably five indicators, the already mentioned m, l and c plus a=environment index and e=age of the patient.

The above-mentioned indexes may have any numerical values, until they are assigned in a congruent and uniform manner, or in other words using the same criteria in the allocation for all the subjects.

For example, the motor index m can be an index of the manipulative capacity of the subject according to a predefined scale. In particular, in the case of patients that are kids such scale can be, among others, the famous “Scala BESTA”, which is a validated assessment tool and standardized for neuroimaging evaluation of the upper limbs for children aged between 6 months and 12 years.

In case the system of the invention is used for other types of training or function as in the functional or cognitive training, the motor index will be determined in alignment with the training desired outcome.

The motor index can always be a simple number. For example, the motor index m can be a number between 0 and 10, or generally a number between 0 and 1.

The other indices can also be defined by a number, for example always from 0 to 10. Initially, for each subject, the values to be assigned to each index may be assigned by a professional operator, for example, depending on the case, a doctor, a therapist, a coach, etc. rather than automatically by the system (i.e. a Machine Learning algorithm)

For example, after a visit and the acquisition of clinical, family, etc. data, the operator (for example a doctor), assigns the initial indexes to each subject (for example a kid) following these rules:

a. motor index: values from 0 to 10 that identify the macro group of motor capabilities (0 means not moving at all a limb, 10 for full capacity)

b. cognitive index: values from 0 to 10 that identify the macro group of cognitive capabilities (0 inability to concentrate, 10 for full capacity)

c. leadership index: values from 0 to 10 that identify the leadership ability (motivation, sense of responsibility, awareness, resilience) of the subject (0 is a pure follower, 10 is a natural leader)

d. environment index: values from 0 to 10 representing the “general well-being” of the family environment.

For example, a subject A could have initial indexes of 7.5.3.2 while another subject B could have initial indexes of 5.7.7.8. The numerical sequence of the indexes for each subject can be defined as the “aggregated initial index”.

In any case, the user database 14 will contain for each subject i-th an association between the subject and the corresponding indexes or indicators, in short bᵢ(m, l, c, a, e) (or other number of indexes, depending on the case).

The part 24 also include a module 25, said “matching module”, which, based on the contents of the user database 14, creates the pairs of users and stores them in the database of the pairs 15, as will be clear from the following.

According to the method of the invention, they are established and stored in a memory 26, of the computerized system, the the coupling rules that define the least permissible deviations between values of two subjects indicators in the user database 14 to be candidates for coupling.

Module 25 leverages such set of rules to find the correspondence between two users which can be mutually paired.

In essence, the simplest rules can advantageously be based on the delta between every index, establishing a maximum deviation or minimum for believing correctly coupling two users A and B. In essence, the coupling function can be schematized the search for the n pairs A, B with (A, B)=f(m, l, c, a, and, Δm, l, c, a, e).

The allowed deviations Δm, Δl, Δ c, Δ a, Δe (or other number of delta depending on the specific case) to be applied can advantageously be contained in the memory 26 together with the rules and be used as input to the coupling module 25 to search for pairs.

For example, given two users A and B, they are paired together to form a torque C (A, B) if:

|m_{A}−m_{B}|=<Δm

|l_{A}−l_{B}|=>Δl

|c_{A}−c_{B}|=<Δc

|a_{A}−a_{B}|=<Δa

|e_{A}−e_{B}|=<Δe

It must be noted that for the leadership index (if present) has been found advantageous that between the two subjects there should be an higher deviation, or one of the two subjects must have a sufficient tendency to leadership greater enough than the other. The other indexes must instead have delta not higher than a certain value.

For example, with the aforementioned values ranging from 0 and 10 for the various indexes, the rules applied by the module 25 can advantageously be the following:
given two subjects A and B, they can be paired together to form a pair C (A, B) if:

|m_{A}−m_{B}|=<Δm=3

|l_{A}−l_{B}|=>Δl=3

|c_{A}−c_{B}|=<Δc=2

|a_{A}−a_{B}|=<Δa=3

|e_{A}−e_{B}|=<Δe=1.5 years for age group 5-9 years; =2 years for age group 10-13.

Of course, the numbers used for the indices can also be a proportion of the numbers described above. For example, if the higher extreme of the indexes is doubled (i.e. to have a greater granularity in the indexes definition, for example, be taken only as integers) also the corresponding offset values will be doubled.

However, the indices may be different in number and type and, of course, any indexes missing for the specific application will not have an assigned gap and will not be used in the formation of the C(A, B) pair.

Thanks to the rules in the memory 26 and to the values of the indicators associated with the subject in the user database it is possible to automatically form pairs of subjects that are in the user database 14 and create in the computer system 10 the pairs database of the pairs 15 formed in such way.

In essence, leveraging the rules in the module 25 it's possible to obtain as output the Cn(A, B) pairs that are stored in the database of the pairs 15.

This process guarantees a first construction of the pairs. In case a subject could be paired with also other subjects, it will be assigned, as example, with the subject with the higher environment value.

If a subject is ineligible for any pair, to pair it, it would be needed to wait that a new suitable subject enters in to the of the system database.

Multiple training or rehabilitation courses can also be planned with different multimedia content, such as different “thematic courses” and the choice could be left to the individual subjects. In this case whenever two subjects are considered suitable to be in the same pair, but they differ for the preference on the course, it must be verified the possibility to converge on the same course (e.g. magic rather than cooking, music, sports or starter to professions or other): if the convergence is not possible the pair cannot be created and we will have to proceed with other pairings or new recruits in mind. Same thing will be done for example for the age of the subjects.

Once established pairs (in whole or in part, as for some subjects in the database 14 may be missed suitable subjects) the system 10 can begin managing the pairs during the means of training or rehabilitation at a distance, starting in the computerized system 10 the training sessions (or rehabilitation) between pairs of subjects in the database of pairs 15 through user terminals 19 available to each subject of the pairs.

The block diagram of the part of the system 10 that can accomplish this is shown in FIG. 3.

For simplicity, in this block diagram only two generic subjects A and B are shown in their corresponding user posts 16, the system being substantially the same for each pair stored in the database 15.

Each subject receives multimedia content, sent from the server 11, which picks them up from the database 13. Such multimedia contents may be of any type useful for the training or rehabilitation and will be produced and selected according to the needs of training or rehabilitation of the single subject. For example, the multimedia contents are developed in order to understand the exercises to be taken for training or rehabilitation, including daily activities and/or thematic topics, in order to keep high the interest of the subject, especially if that child should be encouraged to perform proposing the exercises as a game or in any case, of his/her interest. For example we can think about contents to become a magician, where the tasks contain movements aligned with the rehabilitation plan. The same thing can be done with cooking classes, music etc.

For example, you can identify three basic movements for the upper limbs, namely Reaching, Grasping, Supination (“reach”, “grasp”, “rotate the palm”), and any complex skill is a combination of these three basic movements so that video tutorials can be optimized to keep the focus on these three movements.

Other exercises can be designed for training or development of muscles, tone, reflexes, agility, and so on, as easily imaginable by the expert now.

In any case, the multimedia contents will be displayed on the visualization screen 27 of the user terminal 19, so that the subject can follow what is in them showed and possibly repeating, according to the procedures established by those who have set the path of training or rehabilitation. The server 11 can also select different multimedia contents according to the indexes (for example, ‘m, l, c, a, e’) associated with the subject in the database 14.

For example, the exact media content sent to a subject t will depend on the age and will also be of a suitable type for his motor skills (represented by the index m) and from his cognitive ability (represented by the c index). The content will also depend on the preferences expressed by the individual before the definition of pairs and, of course, from the of training or rehabilitation typology.

Each subject of the torque is captured by the camera 20 associated with post 16 and the image (signal 35 or 36 in FIG. 3) can be likely sent to the other subject of the pair to be displayed on screen 27 for example, in a frame of the screen flanked by the images of the multimedia content, or even at full screen when there is no media to display.

Preferably, even the audio of a subject, detected by the associated microphone 21, can be sent to the other subject to be reproduced by a speaker (not shown) of the corresponding terminal. This allows the two subjects to interact with each other, for example by suggesting the actions to be performed or by showing the other the actions learned from the videos of the multimedia contents transmitted, as will be clear below, or socially interacting. This could generate for example the before mentioned interactions based on training or rehabilitation techniques with the activation of Mirror Neurons.

The images of the two subjects and the multimedia contents can be activated and deactivated from the system using the selection modules 34.

According to the invention, the images taken of each subject of the pair during the sessions allows to automatically establish lm motor or ls stress indexes according to the detected images.

As can be seen in FIG. 3, the camera 20 of each subject is in fact also connected to a module 28 of image processing. The module 28 can detect from the subject a stress level index shot by the camera (for example, if the subject is smiling or laughing, the stress level will be lower than the stress level indicated by a neutral expression or, worst, angry). The module 28 may therefore emits a corresponding stress index ls.

As you will see later, the value or stress index (which can also be defined as an emotional index) can be useful for updating the profile of the individual subject and to change the behavior of the system during the sessions.

In case it is desired, the module 28 may also extrapolate from the image of the subject an index of the level of motor skills of the subject taken by the camera (for example, if the subject succeeds or not to repeat a movement, such as grasping an object, following an exercise learned from the images shown on the screen). The module 28 therefore issues a corresponding index of motor skill lm and can eventually share in real-time these information with the other subject terminal or with the server in order to analyze these data and adapt the system (Emotional Telemetry System).

The module 28 is formed in substance from an image recognition system, with facial recognition functions of expressions and movements of the part of the body (typically the torso with the upper limbs but also the arts and lower limbs). Being such a module type known per se, it will not be here further described or shown in the details, being easily imaginable by the expert technician. The conversion of facial expressions and movements in stress or emotional indexes or motor skill indexes can be realized using for example a library of standardized expressions and movements and each associated with an index value to be assigned.

Module 28 can be implemented in the server, in the user terminal or part in one and in the other. For example, it may be useful preprocessing in the user terminal, in order to reduce the amount of data transmitted to the server with the consequent need of processing data in the server. This is useful especially in the case of a high number of concurrent users.

In function of the motor index lm and or stress for the subject it is possible to elaborate into the computer system 10 new values for that subject of the associated indicators that are useful to establish couplings between subjects.

In particular, the ls index can be sent to a conversion block 29 which transforms ls value in an index by applying a transformation function l=fₗ(ls). The transformation function is selected in such a way as to provide a value in the same scale of the content in the user database values. For example, the function fₗ( ) can simply scale the ls values and return them to a scale from 0 to 10 as already defined above for the leadership indexes, reversing them sign, so that at an high stress index of the subject corresponds to a low index of leadership and vice versa. In fact, it is reasonable that the higher is the stress of the subject, lower will be its ability to lead.

Similarly, if an lm index has been obtained from the image, this lm index will be sent to an optional conversion block 30 which transforms the value lm into m by applying a transformation function m=fₘ (lm). Advantageously, the transformation function is chosen in such a way as to provide a value m in the same scale of m values contained in the user database. For example, the function fₘ( ) can scale the lm values to bring them to a 0 to 10 scale as already defined above for the indexes motor skill.

In essence, the system can obtain from the images of the subject new values for the leadership index and, possibly, for the motor skill index of such subject. This is done for both subjects of each pair and the new values for l and m are compared in a comparison block 31, which receives the data in function of the values lm and/or ls, applying for the comparison the rules of memory 26 already indicated above, in order to verify if the torque C(A, B) is still adequate.

In accordance with the new values calculated by the system for the indicators needed in establishing pairs between subjects, the system can automatically check if the new values of the indexes of the two subjects of the pair still meet the rules stored in memory 26 and in the event of a positive response, maintain the torque.

In case of a negative response, the pair may instead be canceled (either immediately or after a predetermined period of time during which the negative response continues to remain). You can then try to form, according to the rules stored in the memory 26 and the new values of the indicators of the subjects of the canceled pair, a new pair between each subject of the canceled pair and unmatched subjects present in the user database 14.

In essence, in the affirmative case, the system continues with the pair, while in the case of the a result that the indexes are larger than the prefixed A, and therefore the pair is no longer adequate, we wait a time t (e.g. one day), for example defined in a delay block 32 in the output of the comparison block 37.

If the condition of non-adequacy remains for all the time t (and so it's not anymore a temporary condition) the system emit a signal 33 in order to reassign the components of the pair. As will be clear in the following, time t may also vary according to various system conditions.

When the reassignment signal 33 is released the server 11 therefore replaces the new values for m and l of the two subjects of the pair to the previous values in the users database 14 (lines 37 in FIGS. 2 and 3) and control the module 25 in order to determinate new pairs between these subjects and unmatched subjects and that are present in the database 14. In this way, the system adapts dynamically in order to ensure the best possible coupling.

Basically, the torque suitability is established initially from the data inputted in the user database (even only on the basis of established empirical data provided by an operator such as a doctor or therapist) and then is constantly updated depending on the data collected in real time during the remote sessions and keeping always updated the profile of the individual subjects and of the pairs stored in the system.

It is possible in this way to train or re-habilitate “similar” subjects who directly interact in order to mutually improve or emotionally sustain each other.

The subjects are similar in the sense, for example, to have comparable initial capabilities, emotional compatibility, common interests and share the goal of improving their motor or cognitive skills in order to improve their quality of life, and, as indirect result, a greater socialization, self-awareness and confidence, with an overall more active role in their community.

As example, given a set of n children the system creates a set of pairs with children who have similar motor skills and complementary emotional skills, so that the two components of the pair can mutually stimulate their own Mirror Neuron system following a process which includes at first a “mirroring” with a master expert of the subject, then a “mirroring” with themselves reflected in a screen and finally a remote mirroring with a peer.

The good functioning of a pair (toward the shared goal) is then verified in real time and pairs who are inadequate may be reshuffled in order to maintain compatibility of the paired subjects.

To give an example, with the system described is it as if you could do a rehabilitation session together, in the same place, to two children who have similar motor issues but with one of the two that is a bit better than the other and the two that fits from an emotional perspective.

Without the described system, this would not be possible because the probability that two children with these characteristics live at a distance such as to be able to meet and make the rehabilitation together is extremely low. It would also be very expensive, if not almost impossible on a large scale, for the staff in charge of rehabilitation, to continuously monitor the torque in order to verify the maintenance of initial affinity possibly reassign the subjects of the pair (which will lower even more the probability to find other 2 children not yet engaged and available).

By employing this system according to the invention everything becomes automatic and highly reliable and, also, it is possible to work on a large number of children at the same time, with and higher probability to achieve and maintain the most possible effective pairs.

Same goes for any subject who desire to go for any kind of motor/cognitive training, such as for a course of muscle development, psychomotor, job requalification, skill learning etc.

The described system allows you to create the pairs regardless of the physical location of the parties, and also allows you to customize real-time experience of the single session training or rehabilitation according to the specific motor and emotional skills of subjects skills that, as above described, are evaluated at the beginning with an assessment and periodically kept updated through real-time collected data.

With a system aligned to the invention we may also be thinking that the subjects initially examined in validated structures (e.g. hospitals, doctors' offices, training centers, gym etc., depending on the case) that are the most comfortable to reach for the different subjects. In these structures, the initial indexes are determined and entered into a shared database 14 for all facilities.

After that, the system forms the pairs independently from the geographic location of individual subjects and in consideration of the linguistic variable and the components of the pairs are trained to access the system from home based terminals, so that no further physical movement is necessary.

For examples, the subjects are placed in contact, is communicated to them the initial combination in the pair and given all the tools (username and password, the computer, the camera 2d/3d, software, services, etc. or a combination these things) to in order to be able to access to the online platform.

The subjects (or even families in the case of children) agree with each other and keep the system updated with the (StartDate) the preferred time-slot during the day (StartTime) for the daily sessions.

The day of the StartDate, at the StartTime the two parties will have to connect at the same sime to the online platform via their terminals 19, from home via internet and start the training or rehabilitation path that consists, for example, in a monthly course. For example, in the case of rehabilitation of children, the course can be a course to develop the potential of motor, cognitive and social development of the child (e.g. to become a young magician, becoming a chef, a singer, etc.). The course is selected based on the preferences of the subject and of the pair.

The course can be splitter into sub exercises, every one with a similar length, for example on a weekly basis (5-days+2 resting).

Each pre-set period (e.g. every week) the level of challenge in the exercises can increase, or every week we can propose an new task or exercise that can be hard on the first day (because new) but that can be in anyway be done (from motor and/or cognitive perspective) within a pre-established period of time (for example, by the fifth day).

For example, or very day you do one session, lasting for example at the least 30 minutes, consisting of videos and exercises, which are taken from the database of the multimedia content 13.

For example, sessions can be divided into several phases, which can be:
a) Ritual: the subject can be called upon to perform a given exercise, for example, standard for the entire duration of the course, and which allows to measure the motor skills progression and consequently update their profile. The ritual can include established movements useful for the definition of motor skills, for example, inflections of the arm, grasping of objects, etc. (for example in correspondence with the standard motor scales and according to the type of course and the aimed result);
b) Engagement: introduction to the course (1 time), introduction to the exercise of the week (every first day of the week), introduction to the exercise of the day (every day);
c) Video/Content of the exercise. The subject must watch carefully the video of the exercise proposed by the expert/professional of the subject. For the best results each video should have common characteristics such as engaging format and shooting style (for example, for the upper limbs you could have: “Reaching”—Started to side; “Grasping”—Started in front; “Supination”—Started in from above, complete scene—360° shot). Each exercise can still have many versions of the video depending on the the motor ability of the subject that, at operating level playing field, the video of the two parties may be slightly different in in order to activate the best “mirror” mechanism depending on the motor skill of the individual subject, driving the opportunity to personalizing the intervention. Already while the video plays, the system can start recording data from the camera to determine the new motor and leadership indexes as described above, in order to complete or update the profile of the subject.
d) Visualization: the subject can be invited to visualize (with eyes closed eyes) as seen in the previous phase, according to the principle of Motor Imagery, an approach based on the assumption that imagination and execution of the same action share the common activation of some brain circuits. Even here the detection mechanism allows to understand if the subject is relaxed and attentive or distracted and update the appropriate indexes.
e) Self-Mirroring: the subject must repeat alone (or in the case of a child, with the help of the parent/caregiver) the exercise just seen, but looking at himself on the screen of the terminal 16 (preferably on a big screen, like a TV). The system may also propose incentives or “reinforcements” to the correct completion of the exercise and its sub-parts, recording both the motor part that the emotional and continuing to update the relevant indexes in the profile database.

After this first part of the session in which the subject operates mostly alone or with the support of the caregiver, without the other element of the pair, the system moves to the phase of “mirroring” with the other subject of the pair.

In this stage, the two subjects of the torque connect remotely in a kind of video-conference and the aim is to train together, repeat the exercises freshly made in the previous steps. This step is critical both from the point of view of engagement/motivation, than from the point of view of pure motor/cognitive learning processed. The motivation and the “emotional functioning”, the “empathy” between the two (especially if the subjects are children) are critical success factors of the whole process as well as of the pair creation algorithm.

During this phase the system will continue to collect data in order to update the profiles, the telemetry of emotions and verify that coupling conditions and rules are kept valid and, when it's the case, start the process for re-shuffling the pairs as stated in previous sections.

Thanks to the acquisition of the images, it is possible to evaluate the motor performances, cognitive and leadership skills, such as environmental parameters which will then used to update the profiles of subjects and will eventually trigger a change of torque procedure when needed. This can continue throughout the duration of the whole course.

The motor related parameters (i.e. the motor index m) are evaluated as discussed above through the cameras, as well as emotional parameters (joy, anger, engagement, value, frustration etc.) and, reframed in the time interval, contribute to update the subject profile.

At this stage, the emotional parameters can be extended by the simple detection of the individual subject to the interaction between the two parties, for example by estimating a sync of the emotions of the two parties of the pair or the amount of time for propagation of the emotions (Empathy Temeletry or Emotion Telemetry).

The system can estimate the emotions of both subjects and evaluates if they are in the same emotion range at the same time or evaluate the delay needed for the emotion propagation (for example, if one smiles and the other smiles within the next 20 seconds)

This can be used to adapt the function of the blocks 29 which calculate the leadership index of each subject.

For example, it can be determined that is much more relevant to be on the same emotion state rather being both happy as commonly believed, and/or how much one subject has the needed leadership to change the emotions of the other subject (these info of course will update the subject profile).

For example, it may happen that an exercise is difficult and that one of the two subjects is frustrated and sad: even if the other is sad, there is still empathy between the two and this updates the profile of the pair (Empathy Telemetry System).

The mechanism just described can allows a frustration mitigation. For example, if the detection of the index ls stress exceeds a certain value, the system may control the multimedia content to provide positive messages or reinforcement feedbacks (e.g. voiceover, pauses, relaxing music, visual effects, or even, in the case of children, engage the parent/caregiver for a specific action).

After each session, the profile of each individual subject is updated for all the parameters and indexes, as well as the torque profile is updated and the effectiveness of the torque is validated.

As already mentioned above, the adequacy values may also be employed not immediately to reassign a pair but can be estimated on longer period of time, depending on the specific requirements, and the system can in leverage these values applying different weights according to the specific stages of the sessions.

For example, we can establish a weight or “valence”, intended an aggregated index of the session with respect to the exercise. For example, “for a certain number of times the subjects were sad but they are nevertheless committed and have made the exercise” is one situation that will give a good value; “So many times have been happy, but they did not exercise at all” is a situation that will give a low value for the valence of the session.

For example, the valence of the first three days of each week of exercises is a predictive factor the torque effectiveness: if after three days, the value of the valence is low, probably the pair is unlikely to work and then children unlikely will improve, so you have to take action (such as giving tips and advice to parents and verify the fourth day that there is a positive trend of improvement in the valence indicator). If at the end of the fourth or fifth day the value is still low then it is better to finish the week but nonetheless start to reshuffle the torque, or reapply the coupling algorithm with the new parameters collected during these days and recombine the subjects with other peers.

All of these conditions can be part of the function fₘ( ) applied by the block 30 such as of the function fₗ( ) applied by block 29 for each subject of the pairs, as easily imaginable now by the technical expert on the basis of the description already made.

At this point it should be clear how the invention achieves the the goals thanks to a method and a system that enables the coupling of 2 or more subjects and keep updated the couplings efficiently even in situations with a large number of subjects.

In particular, the described system allows optimally to match the subjects, which are for example patients with motor/cognitive deficits, in “online rehabilitative pairs”, and, being available at the “patient home” and low-cost (being based on standard hardware, also in order to facilitate a wide adoption), consent an effective coupling of a large amount of patients, with a minimum weight on traditional rehabilitation facilities and with reduced need in terms of logistic effort, also contributing to a data collection designed to constantly improve the system itself, also providing precious data that the medical community, that now does not collect these information at scale and that has the opportunity to extend its intervention also to domestic scenarios.

The system has proved to be very effective with children in need of functional rehabilitation, although it can also be used effectively with other types of entities and/or other types of training or rehabilitation motor/sensorial/cognitive.

Of course, the above description of embodiments applying the innovative principles of the present invention is given by way of example of such innovative principles and therefore must not be taken as a limitation of the invention claimed herein. For example, the interactions of the subjects of a pair may be different from those described above by way of example.

All the execution system of the described method can be implemented with an appropriate software, in hardware or with a combination of these elements. In specific, the modules in FIGS. 2 and 3 can be implemented via software in the server 12 and/or in the supervisor and user terminals, as easily imaginable by the expert technician. The server and its functional components and databases can also be distributed or in the cloud and therefore placed in various parts of the system and not necessary in a single location.

The timing of the various phases of the method described above may also vary according to the specific cases and types of training and/or rehabilitation, adapting also to specific preferences and practical needs.

1. Method for the coupling of subjects in a computerized system (10) for training or rehabilitation, comprising the steps to:
a) create in the computerized system (10) a user database (14) of subjects to be paired, to each subject, in the user database (14), there are associated values and indexes defined as useful in establishing the couplings between subjects;
b) establish and store in a memory (26) of the computerized system a set of rules that define permissible deviations between the indicator values of two subjects in the user database (14) in order to be paired;
c) automatically forming pairs of subjects in the user database (14) according to the coupling rules stored in the memory (26) and to the values of associated indicators and create in the computerized system (10) a pair database (15) thus formed;
d) start in the computerized system (10) some sessions for training or rehabilitation between pairs of subjects in the pairs database (15) through user terminals (19) available to each subject of the pairs.
e) capture with a camera (20) images of each subject of the pairs during the sessions and automatically calculate motor indexes lm and/or stress ls of the subject according to the detected images;
f) calculating in the computer system (10) as a function of motor indexes lm and/or of stress ls established for the subject, new values for that subject of the associated indicators useful in establishing couplings between subjects;
g) automatically check if the new values of the indicators of the two subjects of the pair still satisfy the rules stored in the memory (26) and in case of positive response keep the torque, while in the event of a negative response cancel the pair and try to train, in according to the rules stored in the memory (26) and the new values of the indicators of the subjects of the canceled torque, a new torque between each subject of the canceled torque and subjects not paired still available in the user database (14);
Repeat necessary steps d) to g).

2. Method according to claim 1, characterized by the fact that indicators comprise at least one motor index, a leadership index, and a cognitive index.

3. Method according to claim 2, characterized by the fact that the indicators also include an environment index and the age of the subject.

4. Method according to claim 2 or 3, characterized in that the indexes have values ranging from 0 to 10.

5. Method according to claim 1, characterized by the fact that the rules to form a pair between two subjects are among these:

|m_{A}−m_{B}|=<Δm

|l_{A}−l_{B}|=>Δl

|c_{A}−c_{B}|=<Δc

|a_{A}−a_{B}|=<Δa

|e_{A}−e_{B}|=<Δe
where m_{A}, m_{B} are indexes of motor skill of the two subjects, l_{A}, l_{B} are indexes of the a leadership of the two subjects, c_{A}, c_{B} are cognitive indexes of the two subjects, a_{A}, a_{B} are the environment indexes the two subjects, and eₐ, e_{B} are ages of the two subjects, and Δm, Δl, Δc, Δa, Δe are their possible differences accepted by the system.

6. Method according to claim 5, characterized by the fact that if the indexes have values ranging from 0 to 10, the acceptable amounts are Δm=3, Δl=3, Δc=2, Δa=3, while Δe=1.5 years for a group in the range of 5-9 years and Δe and =2 years for an group in the range of 10-13 years.

7. Method according to claim 1, characterized by the fact that the pair is reshuffled/dismissed if the negative response detected during step g) lasts for at least a for a period of time t.

8. Computerized system (10) for the coupling of subjects and the execution of sessions of training or rehabilitation between these subjects according to the method of any one of the preceding claims, including a server (11) connected remotely to a plurality of user posts (16), the user database (14), the database of the pairs (15) and the memory (26).

9. Computerized system according to claim 8, characterized by the fact that the user posts (16) comprise a user terminal (19) and the camera (20) with the shooting of a subject of the pair during a session.

10. Computerized system according to claim 8, characterized by the fact that includes a coupling module (25) connected to the user database (14) and to the memory (26) to form the pairs of users based on the user database content (14) and rules taken from the memory (26) and stored in a database of pairs (15).

11. Computerized system according to claim 8, characterized by the fact that includes also a database (13) that contains media content to display on a screen (27) of the user posts (16).

12. Computerized system according to claim 8, characterized by the fact that includes an image processing modules (28) which receive the image from the cameras (20) and calculate therefrom said motor (lm) and/or stress (ls) indexes of the subject based on the detected images.

13. Computerized system according to claim 8, characterized by the fact of including a comparison block (31) that receives data as a function of the said indexes lm and/or ls and compares them according to the rules in memory (26) to eventually delete the pair and create a new pair.

14. Computer system according to claim 8, characterized by the fact that the image taken by a camera (20) of a subject of a pair is sent to a screen (27) at the user post (16) of the other subject of the pair.

15. Computerized system according to claim 8, characterized by the fact that the user posts (16) and the server (11) are connected through a network (18), ideally the Internet.
